(19) United States
(12) Patent Application Publication
(10) Pub. No.: US 2026/0125434 A1
(43) Pub. Date: May 7, 2026

(54) CXCL-BPI FUSION PROTEIN AND USE THEREOF

(71) Applicants: BEIJING ANJUN GENETECH CO., LTD., Beijing (CN); XIAMEN LIANHEANJIN BIOTECH CO., LTD., Fujian (CN)

(72) Inventors: Jindong CHEN, Beijing (CN); Miqing YANG, Fujian (CN); Chao XING, Beijing (CN); Jingchao LI, Fujian (CN); Wei CAO, Beijing (CN); Bin CAO, Beijing (CN); Yunqing AN, Fujian (CN)

(21) Appl. No.: 19/114,523

(22) PCT Filed: May 26, 2023
(86) PCT No.: PCT/CN2023/096553

§ 371 Date: Mar. 24, 2025

(30) Foreign Application Priority Data
Sep. 28, 2022 (CN) 202211196880.5

(51) Int. Cl. C07K 14/47; A61K 38/00; A61P 31/04; C07K 14/52; C12N 15/62; C12N 15/85

(52) U.S. Cl. CPC C07K 14/4742; A61P 31/04; C07K 14/522; C12N 15/62; C12N 15/85; A61K 38/00; C07K 2319/00

(57) ABSTRACT

The present invention discloses a CXCL-BPI fusion protein that can be used for treating Gram-negative bacterial infections, a coding nucleic acid thereof, a method for expressing and preparing the same, and a use thereof in the manufacture of a pharmaceutical composition for treating Gram-negative bacterial infections. The CXCL-BPI fusion protein comprises a human ELR+CXC chemokine and a bioactive fragment of N-terminal domain of human BPI, and has the dual functions of both ELR+CXC chemokine and BPI, with ability of binding to LPS and directly killing Gram-negative bacteria, and also inducing chemotaxis and promoting phagocytes to target, bind to and phagocytize Gram-negative bacteria. The mechanism of action of the fusion protein can overcome Gram-negative bacterial drug resistance.

### CROSS REFERENCE TO RELATED APPLICATION

This application claims priority to a Chinese patent application with No. 202211196880.5 filed on Sep. 28, 2022, incorporated by reference herein in its entirety.

### TECHNICAL FIELD

The present invention relates to the field of biomedicine. Specifically, the present invention relates to a CXCL-BPI fusion protein that can be used for treating Gram-negative bacterial infections, a coding nucleic acid thereof, a method for expressing and preparing the same, and use thereof in the manufacture of a pharmaceutical composition for treating Gram-negative bacterial infections. The CXCL-BPI fusion protein comprises a human ELR+CXC chemokine and a bioactive fragment of N-terminal domain of human BPI, has the dual functions of both ELR+CXC chemokine and BPI, with ability of binding to LPS and directly killing Gram-negative bacteria, and also inducing chemotaxis and promoting phagocytes to target, bind to and phagocytize Gram-negative bacteria, and the mechanism of action of the fusion protein can overcome Gram-negative bacterial drug resistance.

### BACKGROUND ART

Chemokines are a class of family of cytokines with small molecular weight that have chemotaxis on cells (especially leukocytes); and chemokine receptors (CKRs) are a class of superfamily of G protein-coupled receptors (GPCRs) that mediate chemokines to exert functions. Chemokines can mediate the migration of immune cells to sites of infection and inflammation, and stimulate immune cells to participate in immune responses and inflammation. Phagocytes such as granulocytes and monocytes/macrophages in higher animals have phagocytosis and bactericidal activities and are an important part of nonspecific immunity in body. Chemokines and phagocytes play an important role in the immunity against bacterial infections in body, but they are not characterized by a specific mechanism of action similar to the opsonization of specific IgG antibodies to facilitate the phagocytosis of bacteria. Chemokines are divided into four subfamilies, CC, CXC, CX3C and C, according to the arrangement of the two cysteines near the N-terminus, wherein the CXC subfamily is divided into ELR+CXC chemokines and non-ELR+CXC chemokines according to whether there is an ELR (Glu-Leu-Arg) motif structure before the first Cys. From 1986 to the early 1990s, IL-8, CXCR1 and CXCR2 (also known as IL-8RA and IL-8RB) and their ELR+CXC chemokine ligands were discovered successively. CXCR1 and CXCR2 are G protein-coupled receptors (GPCRs) mainly expressed in cells such as neutrophils, monocytes/macrophages and endothelial cells. The ligands of CXCR1 and CXCR2 constitute the ELR+CXC chemokine family. The seven known human ELR+CXC chemokines are Gro-α (also known as CXCL1), Gro-O (CXCL2), Gro-γ (CXCL3), ENA-78 (CXCL5), GCP-2 (CXCL6), NAP-2 (CXCL7) and IL-8 (CXCL8), and main functions thereof are to induce chemotaxis of neutrophils mainly (as well as monocytes/macrophages) and promote angiogenesis; wherein, all seven chemokines (CXCL1, CXCL2, CXCL3, CXCL5, CXCL6, CXCL7 and CXCL8) mediate signals by binding to CXCR2, while CXCL6 and CXCL8 also mediate signals by binding to CXCR1 (all of which contribute to the redundancy of activation pathways). IL-8 (CXCL8) is a representative member of the CXC subfamily, the main functions thereof are to induce chemotaxis and stimulate phagocytosis of neutrophils mainly, as well as monocytes/macrophages to some extent. See, for example, [Hughes, et al., FEBS J. (2018) 285(16): 2944][Bacon, et al., J. Interferon Cytokine Res. (2002) 22(10): 1067][Bi Huijuan et al., Journal of Immunology (2010) 26(12): 1091][Baggiolini et al., Adv Immunol. (1993) 55: 97].

Bactericidal/permeability-increasing protein (BPI) is a ˜55 kDa cationic antimicrobial protein composed of 456 amino acid residues, which was first discovered in human polymorphonuclear neutrophils by Weiss et al in 1978, and the structure of the protein consists of an N-terminal domain and a C-terminal domain connected via a domain linker. Studies have confirmed that the bioactive fragments of N-terminal domain BPI₁₋₁₉₉ and BPI₁₋₁₉₃ (which are the fragment of amino acid residues 1-199 of the N-terminal domain, and the fragment of amino acid residues 1-193 of the N-terminal domain that is truncated by 6 amino acids at the C-terminal of BPI₁₋₁₉₉, respectively) exhibit the same activities as human holo-BPI to highly bind to lipopolysaccharide (LPS) and lipid A of Gram-negative bacterium (GNB), neutralize endotoxin, and increase the envelope permeability of susceptible GNB, thereby directly killing GNB. XOMA has developed the recombinant bioactive fragment of N-terminal domain of human BPI (NEUPREX@rBPI21) since the 1990s, and has conducted a number of clinical trials. However, due to the fact that a high concentration of BPI needs to be maintained for a long period of time to kill bacteria, and the rBPI21 has a short half-life in vivo, a large therapeutic dose thereof is needed, and an effective therapeutic concentration thereof is difficult to be maintained in the body, the rBPI21 has not achieved clinical success and has not been approved by the FDA. See, for example, [Weiss, et al., J. Biol. Chem. (1978) 253: 2664][Gray, et al., J Biol Chem. (1989) 264: 9505][Kleiger, et al., J. Mol. Biol. (2000) 299: 1019][Elsbach, J. Leukoc. Biol. (1998) 64: 14][Levin, et al., Lancet. (2000) 356: 961][Giroir, et al., Crit Care Med (2001) 29(7) (Suppl.): S130].

Gram-negative bacteria are one of the most main pathogens causing infectious diseases and highly resistant to commonly used clinical antibiotics, and they are drug-resistant pathogens most concerned at the current. In February 2017, WHO published its first ever list of 12 families of drug-resistant bacteria that pose the greatest threat to human health, of which 9 are Gram-negative bacteria. About half of patients with bacterial infections are infected with Gram-negative bacteria, among which severe infections with Gram-negative bacteria will lead to sepsis, even causing endotoxic shock and death, for which there is still no effective treatment.

Therefore, an object of the present invention is to utilize the biological characteristics of ELR+CXC chemokines and BPI to provide a CXCL-BPI fusion protein that can be used to treat Gram-negative bacterial infections, and the mechanism of action thereof can overcome Gram-negative bacterial drug resistance.

### Contents of the Present Invention

### Brief Description of the Invention

It is an object of the present invention to provide a fusion protein comprising a chemokine and BPI, which has the dual functions of both chemokine and BPI, with ability of binding to LPS and directly killing Gram-negative bacteria, and also inducing chemotaxis and promoting phagocytes to target, bind to and phagocytize Gram-negative bacteria (which is featured by a specific mechanism of action), and the mechanism of action of the fusion protein can overcome Gram-negative bacterial drug resistance, which can be used to treat Gram-negative bacterial infections and prepare a pharmaceutical composition for treating Gram-negative bacterial infections.

In one aspect of the present invention, there is provided a fusion protein comprising a chemokine and BPI that can be used to treat a Gram-negative bacterial infection, and a coding nucleic acid thereof, which, optionally, wherein the coding nucleic acid is a coding DNA.

Specifically, the chemokine in the present invention is preferably a human ELR+CXC chemokine, and the BPI is preferably a bioactive fragment of N-terminal domain of human BPI, thereby providing a CXCL-BPI fusion protein comprising a human ELR+CXC chemokine and a bioactive fragment of N-terminal domain of human BPI.

In another aspect of the present invention, there is provided a coding nucleic acid of the CXCL-BPI fusion protein, and an efficient expression vector thereof, a host cell stably transfected with and expressing the same, as well as a method of extracting and preparing the same, optionally, wherein the coding nucleic acid is a coding DNA.

In yet another aspect of the present invention, there is provided the use of the CXCL-BPI fusion protein for treating a Gram-negative bacterial infection, and/or use thereof in the manufacture of a pharmaceutical composition for treating a Gram-negative bacterial infection.

### DETAILED DESCRIPTION OF THE INVENTION

The objects and implementation of the present invention are further described below, and the scopes, contents and advantages of the present invention are obvious to those skilled in the art. Although the present invention provides preferred embodiments, those skilled in the art will recognize that various modifications and variations are also within the scopes of the present invention. Therefore, the appended claims and any future amendments and changes thereto are within the scopes.

Chemokines can mediate the migration of immune cells to sites of infection and inflammation, stimulate immune cells to participate in immune response and inflammation; phagocytes such as granulocytes, monocytes/macrophages in higher animals have phagocytosis and bactericidal activities and are an important part of nonspecific immunity in body. Chemokines and phagocytes play an important role in the immunity against bacterial infections in body, but they are not characterized by a specific mechanism of action similar to the opsonization of specific IgG antibodies to facilitate the phagocytosis of bacteria.

CXCR1 and CXCR2 are G protein-coupled receptors (GPCRs) mainly expressed in cells such as neutrophils, monocytes/macrophages and endothelial cells. The ligands of CXCR1 and CXCR2 constitute the ELR+CXC chemokine family, and main functions thereof are to induce chemotaxis of neutrophils maintly (as well as monocytes/macrophages) and promote angiogenesis, which biological activities are not species-specific. IL-8 (CXCL8) is a representative member of the CXC subfamily, the main functions thereof are to induce chemotaxis and stimulate phagocytosis of neutrophils maintly, as well as monocytes/macrophages to some extent.

The bioactive fragment of N-terminal domain of human BPI has the same activities as human holo-BPI to highly bind to lipopolysaccharide (LPS) and lipid A of Gram-negative bacterium (GNB), neutralizes endotoxin, and increases the envelope permeability of susceptible GNB, thereby directly killing GNB. However, due to the fact that a high concentration of a BPI needs to be maintained for a long period of time to kill bacteria when the BPI is used alone and it is difficult to maintain an effective therapeutic concentration of BPI in vivo, BPI has not yet achieved clinical success.

To this end, in one aspect of the present invention, there is provided a fusion protein comprising a chemokine and a BPI; specifically, the chemokine is preferably a human ELR+CXC chemokine that can function through the mediation of CXCR1 and CXCR2 expressed in neutrophils, monocytes/macrophages and endothelial cells, and the BPI is preferably a bioactive fragment of N-terminal domain of human BPI, thereby providing a CXCL-BPI fusion protein comprising a human ELR+CXC chemokine and a bioactive fragment of N-terminal domain of human BPI. The human ELR+CXC chemokine is selected from the group consisting of CXCL1, CXCL2, CXCL3, CXCL5, CXCL6, CXCL7 and CXCL8 (collectively referred to as CXCL), and the bioactive fragment of N-terminal domain of human BPI is selected from the group consisting of BPI₁₋₂₃₃ (a fragment of amino acid residues 1-233 of the N-terminal domain), BPI₁₋₁₉₉ and BPI₁₋₁₉₃ of the N-terminal domain. In a specific embodiment, the CXCL1-BPI, CXCL2-BPI, CXCL3-BPI, CXCL5-BPI, CXCL6-BPI, CXCL7-BPI and CXCL8-BPI fusion proteins (collectively referred to as CXCL-BPI) are designed and constructed, wherein, a human ELR+CXC chemokine is preferably used as the N-terminal domain of the fusion protein, and the BPI₁₋₂₃₃ of N-terminal domain of human BPI is preferably used as the C-terminal domain of the fusion protein, and preferably, the two are connected via a linker, and the fusion protein comprises, from the N-terminus to the C-terminus, a CXCL, a linker, and a BPI₁₋₂₃₃ sequence as elements in sequence.

In another aspect of the present invention, there is provided a coding DNA sequence of the CXCL-BPI fusion protein, and an efficient expression vector thereof, a host cell stably transfected with and expressing the same, as well as a method of extracting and preparing the same. Specifically, the present invention designs and optimizes the coding DNA sequence of the CXCL-BPI fusion protein, which comprises, from the 5′ end to the 3′ end, a 5′ end adapter sequence (containing an EcoR I restriction site), a signal peptide coding sequence, a CXCL coding sequence, a linker coding sequence, a BPI₁₋₂₃₃ coding sequence, and a 3′ end adapter sequence (containing a TGA termination codon and a Sal I restriction site) as elements in sequence; and the present invention constructs an efficient expression vector, whereby a CXCL-BPI fusion protein is stably transformed, efficiently expressed, extracted and prepared in mammalian cells.

The present invention confirms that the CXCL-BPI fusion protein has the dual functions of both ELR+CXC chemokine and BPI, with ability of binding to LPS and directly killing Gram-negative bacteria, and also inducing chemotaxis and promoting phagocytes to target, bind to and phagocytize Gram-negative bacteria (which is featured by a specific mechanism of action), and the mechanism of action of the fusion protein can overcome Gram-negative bacterial drug resistance; and, the CXCL-BPI fusion protein has a significant bactericidal effect in peripheral blood and peritoneal phagocytes, and has a significant protective effect on mice infected with Gram-negative bacteria. Therefore, in another aspect of the present invention, the CXCL-BPI fusion protein can be used to treat Gram-negative bacterial infections and in the manufacture of a pharmaceutical composition for treating Gram-negative bacterial infections.

Gram-negative bacteria are one of the most main pathogens causing infectious diseases and highly resistant to commonly used clinical antibiotics, and they are drug-resistant pathogens most concerned at the current. In February 2017, WHO published its first ever list of 12 families of drug-resistant bacteria that pose the greatest threat to human health, of which 9 are Gram-negative bacteria. About half of patients with bacterial infections are infected with Gram-negative bacteria, among which severe infections with Gram-negative bacteria will progress to sepsis, causing endotoxic shock and death, for which there is still no effective treatment. The CXCL-BPI fusion protein described in the present invention is useful in treating Gram-negative bacterial infections and has great clinical needs and application prospects.

Therefore, in one aspect, the present invention provides a CXCL-BPI fusion protein comprising a human ELR+CXC chemokine and a bioactive fragment of N-terminal domain of human BPI.

In one embodiment, the human ELR+CXC chemokine in the fusion protein is selected from the group consisting of human CXCL1, human CXCL2, human CXCL3, human CXCL5, human CXCL6, human CXCL7 and human CXCL8, optionally, wherein the human CXCL8, human CXCL1, human CXCL2, human CXCL3, human CXCL5, human CXCL6 or human CXCL7 comprises the sequence as set forth in SEQ ID NO: 1, SEQ ID NO: 2, SEQ ID NO: 3, SEQ ID NO: 4, SEQ ID NO: 5, SEQ ID NO: 6 or SEQ ID NO: 7, respectively.

In another embodiment, the bioactive fragment of N-terminal domain of human BPI in the fusion protein is selected from the group consisting of human BPI₁₋₂₃₃, BPI₁₋₁₉₉ and BPI₁₋₁₉₃, optionally, wherein the human BPI₁₋₂₃₃ comprises the sequence as set forth in SEQ ID NO: 10.

In yet another embodiment, the human ELR+CXC chemokine is used as the N-terminal domain of a fusion protein, and the bioactive fragment of N-terminal domain of human BPI is used as the C-terminal domain of the fusion protein, and the two are optionally connected via a linker, and further optionally, the linker is selected from GPPSGSGGGSGGG (SEQ ID NO: 8) and GGGSGGGSGGG (SEQ ID NO: 9).

In another aspect, the present invention provides a nucleic acid, which encodes the CXCL-BPI fusion protein of the present invention.

In one embodiment, the nucleic acid comprises, from the 5′ end to the 3′ end, a 5′ end adapter sequence, a signal peptide coding sequence, a human ELR+CXC chemokine coding sequence, a linker coding sequence, a coding sequence of the bioactive fragment of N-terminal domain of human BPI and a 3′ end adapter sequence in sequence, optionally, wherein the human ELR+CXC chemokine coding sequence comprises the sequence as set forth in SEQ ID NO: 13, SEQ ID NO: 14, SEQ ID NO: 15, SEQ ID NO: 16, SEQ ID NO: 17, SEQ ID NO: 18 or SEQ ID NO: 19, respectively, and the coding sequence of the bioactive fragment of N-terminal domain of human BPI comprises the sequence as set forth in SEQ ID NO: 22.

In another aspect, the present invention provides the use of the CXCL-BPI fusion protein of the present invention for treating a Gram-negative bacterial infection, and/or use thereof in the manufacture of a pharmaceutical composition for treating a Gram-negative bacterial infection.

In another aspect, the present invention provides an expression vector, which is useful for expressing the CXCL-BPI fusion protein of the present invention, optionally, the expression vector comprises a nucleic acid encoding the CXCL-BPI fusion protein of the present invention.

In one embodiment, the expression vector is selected from the group consisting of efficient expression vectors pSCm-CXCLT-BPI, pSCm-CXCL2-BPI, pSCm-CXCL3-BPI, pSCm-CXCL5-BPI, pSCm-CXCL6-BPI, pSCm-CXCL7-BPI, and pSCm-CXCL8-BPI.

In another aspect, the present invention provides a pharmaceutical composition comprising the CXCL-BPI fusion protein of the present invention and a pharmaceutically acceptable carrier.

In another aspect, the present invention provides a host cell comprising an expression vector, wherein the expression vector is capable of performing a stable transfection or transformation with a nucleic acid encoding the CXCL-BPI fusion protein of the present invention.

In another aspect, the present invention provides a method for preparing the CXCL-BPI fusion protein of the present invention, comprising culturing the host cell of the present invention under conditions suitable for the expression of the CXCL-BPI fusion protein, harvesting the expressed CXCL-BPI fusion protein, and optionally further purifying the expressed CXCL-BPI fusion protein.

In another aspect, the present invention provides a method for treating Gram-negative bacterial infection, comprising administering a therapeutically effective amount of the CXCL-BPI fusion protein or pharmaceutical composition of the present invention to a subject suffering from a Gram-negative bacterial infection.

In one embodiment, the method for treating Gram-negative bacterial infection of the present invention further comprises administering an antibiotic compound to the subject suffering from a Gram-negative bacterial infection prior to, concurrent, or after administration of a therapeutically effective amount of the CXCL-BPI fusion protein or pharmaceutical composition of the present invention.

### BRIEF DESCRIPTION OF THE DRAWINGS

FIG. 1 shows the extraction and preparation of CXCL-BPI fusion protein. Wherein: Figure TA shows the typical spectrum of SP Sepharose® Fast Flow cation exchange chromatography;

FIG. 1B shows the SDS-PAGE electrophoresis of purified proteins of interest.

FIG. 2 shows that CXCL-BPI fusion proteins bind to endotoxin.

FIG. 3 shows that CXCL-BPI fusion proteins directly kill Gram-negative bacteria.

FIG. 4 shows that CXCL-BPI fusion proteins induce chemotaxis of human HL-60 cells.

FIG. 5 shows that CXCL-BPI fusion proteins induce chemotaxis of mouse bone marrow neutrophils.

FIG. 6 shows that CXCL-BPI fusion proteins induce chemotaxis of mouse peritoneal cells.

FIG. 7 shows that CXCL-BPI fusion proteins promote phagocytes to target, bind to and phagocytize Gram-negative bacteria. Wherein: A/B: human HL-60 cells; C: human peripheral blood leukocytes; D: mouse peripheral blood leukocytes; E: mouse peritoneal phagocytes.

FIG. 8 shows the bactericidal effect of CXCL-BPI fusion proteins in human/mouse peripheral blood.

FIG. 9 shows the bactericidal effect of CXCL-BPI fusion proteins in mouse peripheral blood.

FIG. 10 shows the bactericidal effect of CXCL-BPI fusion proteins in mouse peritoneal phagocytes.

FIG. 11 shows the protective effect of CXCL-BPI fusion proteins on mice infected with Gram-negative bacteria. A: mouse infection model (dose); B: serum; C: liver; D: spleen.

### BIOLOGICAL DEPOSIT INFORMATION

The present invention relates to the Escherichia coli deposited in the China General Microbiological Culture Collection Center on Sep. 15, 2022, with a deposit number of CGMCC NO.: 25726 and a name of pSCm-IL8-BPI (in E. coli JM108).

### Specific Models for Carrying Out the Present Invention

The technical solution of the present invention is further described in conjunction with the examples and drawings below, but is not limited to the examples of the present application. More specifically, Example 1 relates to a CXCL-BPI fusion protein and a coding DNA sequence thereof as well as expression and preparation thereof; Example 2 relates to the biological function of a CXCL-BPI fusion protein, with ability of binding to LPS and directly killing Gram-negative bacteria, and also inducing chemotaxis and promoting phagocytes to target, bind to and phagocytize Gram-negative bacteria; Example 3 relates to the bactericidal effect of CXCL-BPI fusion proteins in peripheral blood and peritoneal phagocytes; and Example 4 relates to the protective effect of CXCL-BPI fusion proteins on mice infected with Gram-negative bacteria. The scopes, contents and advantages of these examples are obvious, and various modifications and variations are also within the scopes of the present description, which include but are not limited to a CXCL-BPI fusion protein and its coding DNA sequence, as well as other equivalents, isoforms, variants and analogues of each of elements.

### Example 1: CXCL-BPI Fusion Protein and Coding DNA Sequence Thereof, Expression and Preparation Thereof

### 1. CXCL-BPI Fusion Protein and Coding DNA Sequence Thereof

In the present invention, a CXCL-BPI fusion protein was designed and constructed, which comprised, from N-terminal to C-terminal, a CXCL, a linker, and a BPI₁₋₂₃₃ sequence as elements (as shown in Table 1). In the present invention, the coding DNA sequence of the CXCL-BPI fusion protein was designed and optimized, which comprised, from the 5′ end to the 3′ end, a 5′ end adapter sequence (containing an EcoR I restriction site), a signal peptide coding sequence, a CXCL coding sequence, a linker coding sequence, a BPI₁₋₂₃₃ coding sequence, and a 3′ end adapter sequence (containing a TGA termination codon and a Sal I restriction site) as elements (as shown in Table 2).

TABLE 1
Composition of CXCL-BPI fusion protein

| CXCL- | | | |
|---|---|---|---|
| BPI | CXCL | Linker | BPI₁₋₂₃₃ |
| CXCL8- | SAKELRCQCIKTYSKPFHPKFIKELRVIESG | GPPSGSG | VNPGVVVRISQK |
| BPI | PHCANTEIIVKLSDGRELCLDPKENWVQR | GGSGGG | GLDYASQQGTAA |
| | VVEKFLKRAE (wherein the two amino acid | (SEQ ID | LQKELKRIKIPDY |
| | residues NS at the C-terminal were truncated) | NO: 8) | SDSFKIKHLGKG |
| | (SEQ ID NO: 1) | | HYSFYSMDIREF |
| CXCL1- | ASVATELRCQCLQTLQGIHPKNIQSVNVKS | GGGSGG | QLPSSQISMVPNV |
| BPI | PGPHCAQTEVIATLKNGRKACLNPASPIVK | GSGGG | GLKFSISNANIKIS |
| | KIIEKMLNSDKSN (SEQ ID NO: 2) | (SEQ ID | GKWKAQKRFLK |
| CXCL2- | APLATELRCQCLQTLQGIHLKNIQSVKVKS | NO: 9) | MSGNFDLSIEGM |
| BPI | PGPHCAQTEVIATLKNGQKACLNPASPMV | | SISADLKLGSNPT |
| | KKIIEKMLKNGKSN (SEQ ID NO: 3) | | SGKPTITASSCSS |
| CXCL3- | ASVVTELRCQCLQTLQGIHLKNIQSVNVR | | HINSVHVHISKSK |
| BPI | SPGPHCAQTEVIATLKNGKKACLNPASPM | | VGWLIQLFHKKI |
| | VQKIIEKILNKGSTN (SEQ ID NO: 4) | | ESALRNKMNSQV |
| CXCL5- | LRELRCVCLQTTQGVHPKMISNLQVFAIGP | | CEKVTNSVSSEL |
| BPI | QCSKVEVVASLKNGKEICLDPEAPFLKKVI | | QPYFQTLPVMTK |
| | QKILDGGNKEN (SEQ ID NO: 5) | | IDSVAGINYGLVA |
| CXCL6- | VLTELRCTCLRVTLRVNPKTIGKLQVFPAG | | PPATTAETLDVQ |
| BPI | PQCSKVEVVASLKNGKQVCLDPEAPFLKK | | MKGEFYSENH |
| | VIQKILDSGNKKN (SEQ ID NO: 6) | | (amino acid residue |
| CXCL7- | AELRCMCIKTTSGIHPKNIQSLEVIGKGTH | | 132 was changed |
| BPI | CNQVEVIATLKDGRKICLDPDAPRIKKIVQ | | from C to A)(SEQ |
| | KKLAGDESAD (SEQ ID NO: 7) | | (ID NO:10) |

TABLE 2
Composition of coding DNA sequence of CXCL-BPI fusion protein

| | | Signal | | | | |
|---|---|---|---|---|---|---|
| | 5′ end | peptide | | Linker | BPI1-233 | 3′ end |
| CXCL- | adaptor | coding | | coding | coding | adaptor |
| BPI | sequence | sequence | CXCL coding sequence | sequence | sequence | sequence |
| CXCL8- | gaattcgc | atgggatgga | tctgccaaggaactgcggtgtcagtgcatcaaga | ggacctccaagt | gtgaaccccggc | tgagtcgac |
| BPI | cacc | gttgcatcat | cctacagcaagcctttccaccccaagttcatcaa | ggcagtggtgg | gtggtggtgcgg | (SEQ ID |
| | (SEQ ID | cctgtttctg | agaactgagagtgatcgagtccggccctcactgt | cggatccggtgg | atctcccagaaa | NO: 23) |
| | NO: 11) | gtggctaccg | gccaacaccgagatcatcgtgaagctgtccgacg | cggt (SEQ | ggcctggattac | |
| | | caacaggcgt | gccgcgagctgtgcctggaccctaaggaaaattg | ID NO: 20) | gccagccagca | |
| | | gcattct | ggtgcagagagtcgtggaaaagtttctgaagaga | | gggaacagctgc | |
| | | (SEQ ID | gccgag (SEQ ID NO: 13) | | tctgcagaaaga | |
| CXCL1- | | NO: 12) | gcttctgtcgccaccgagctgcggtgccagtgcc | ggtggaggcag | actgaaaagaat | |
| BPI | | | tgcagaccctgcagggcatccaccccaagaaca | tggtggcggatc | caagatcccaga | |
| | | | tccagtccgtgaacgtgaaatctcctggccctca | cggtggcggt | ctacagtgatagc | |
| | | | ctgcgcccagaccgaagtgatcgccacactgaag | (SEQ ID | ttcaagatcaagc | |
| | | | aacggccggaaggcctgtctgaaccctgcctctc | NO: 21) | acctgggaaagg | |
| | | | caatcgtgaagaagatcatcgagaagatgctgaa | | gccactactctttc | |
| | | | ctccgacaagtccaac (SEQ ID NO: 14) | | tactctatggacat | |
| CXCL2- | | | gctcctctggctaccgagctgcggtgccagtgcc | | cagagagtttcag | |
| BPI | | | tgcagaccctgcagggcatccacctgaagaacat | | ctgccctcctctc | |
| | | | ccagtccgtgaaagtgaagtctccaggccctcac | | agatctctatggt | |
| | | | tgcgcccagaccgaagtgatcgccacactgaag | | gcctaacgtggg | |
| | | | aacggacagaaggcctgtctgaaccccgcctcc | | cctgaagttcagc | |
| | | | cctatggtcaagaagatcatcgagaagatgctga | | atctccaacgcca | |
| | | | aaaacggcaagtccaac (SEQ ID NO: 15) | | atatcaagatttct | |
| CXCL3- | | | gcttctgtcgtgaccgagctgagatgccagtgcct | | ggcaagtggaag | |
| BPI | | | gcagacactgcagggcatccacctgaaaaacat | | gctcagaagcgg | |
| | | | ccagtccgtgaacgtgcggtctccaggacctcac | | ttcctgaagatgt | |
| | | | tgcgcccagaccgaagtgatcgccaccctgaag | | ctggcaacttcga | |
| | | | aacggcaagaaggcctgtctgaatcctgctagcc | | cctgtccatcgaa | |
| | | | ctatggtgcagaagatcatcgagaagatcctgaa | | ggcatgtccatca | |
| | | | caagggctccaccaac (SEQ ID NO: 16) | | gcgccgatctga | |
| CXCL5- | | | ctgagagagctgcggtgcgtgtgtctgcagacca | | agctgggctctaa | |
| BPI | | | cacagggcgtgcaccctaagatgatctccaacct | | tcctacctccggc | |
| | | | gcaggtgttcgccatcggccctcagtgctccaag | | aagcccacaatc | |
| | | | gtggaagtggtggcctccctgaagaacggcaag | | accgcctctagct | |
| | | | gagatctgcctggaccctgaggcccctttcctgaa | | gttcctctcacatc | |
| | | | gaaagtgatccagaagatcctggacggcggcaa | | aacagcgtgcac | |
| | | | caaggaaaat (SEQ ID NO: 17) | | gtgcacatctcta | |
| CXCL6- | | | gtgctgaccgagctgagatgcacctgtctgagag | | agtccaaagtgg | |
| BPI | | | tgaccctgcgggtgaaccccaagaccatcggca | | gctggctgatcca | |
| | | | agctgcaggtgttccctgctggccctcagtgctcc | | gctgttccacaag | |
| | | | aaggtggaagtggtggcctctctgaagaacggc | | aagatcgagtctg | |
| | | | aaacaggtgtgcctggaccctgaggctcctttcct | | ctctgcggaaca | |
| | | | gaagaaagtgatccagaagatcctggactccgg | | agatgaactctca | |
| | | | caacaagaagaat (SEQ ID NO: 18) | | ggtgtgcgagaa | |
| CXCL7- | | | gccgagctgagatgcatgtgcatcaagaccacct | | ggtgaccaactc | |
| BPI | | | ctggcatccatcccaagaacatccagtccctgga | | cgtgtccagcga | |
| | | | agtcatcggcaagggaacccactgcaaccaggt | | actccagccttatt | |
| | | | ggaagtgatcgccacactgaaggacggcagaaa | | tccagaccctgc | |
| | | | gatctgcctggaccctgacgctcctcggatcaag | | ccgtgatgacca | |
| | | | aaaatcgtgcagaagaagctggctggcgacgag | | agatcgactccgt | |
| | | | tccgctgat (SEQ ID NO: 19) | | ggctggcatcaa | |
| | | | | | ctacggcctggt | |
| | | | | | ggccccacctgc | |
| | | | | | tactaccgccga | |
| | | | | | gacactggacgt | |
| | | | | | gcagatgaaggg | |
| | | | | | cgagttctactcc | |
| | | | | | gagaaccac | |
| | | | | | (SEQ ID NO: | |
| | | | | | 22) | |

The sequences of CXCL1-BPI, CXCL2-BPI, CXCL3-BPI, CXCL5-BPI, CXCL6-BPI, CXCL7-BPI and CXCL8-BPI in the CXCL-BPI fusion protein were as follows:

CXCL1-BPI:
(SEQ ID NO: 24)
ASVATELRCQCLQTLQGIHPKNIQSVNVKSPGPHCAQTEVIATLKNGRKA
CLNPASPIVKKIIEKMLNSDKSNGGGSGGGSGGGVNPGVVVRISQKGLDY
ASQQGTAALQKELKRIKIPDYSDSFKIKHLGKGHYSFYSMDIREFQLPSS
QISMVPNVGLKFSISNANIKISGKWKAQKRFLKMSGNFDLSIEGMSISAD
LKLGSNPTSGKPTITASSCSSHINSVHVHISKSKVGWLIQLFHKKIESAL
RNKMNSQVCEKVTNSVSSELQPYFQTLPVMTKIDSVAGINYGLVAPPATT
AETLDVQMKGEFYSENH
CXCL2-BPI:
(SEQ ID NO: 25)
APLATELRCQCLQTLQGIHLKNIQSVKVKSPGPHCAQTEVIATLKNGQKA
CLNPASPMVKKIIEKMLKNGKSNGGGSGGGSGGGVNPGVVVRISQKGLDY
ASQQGTAALQKELKRIKIPDYSDSFKIKHLGKGHYSFYSMDIREFQLPSS
QISMVPNVGLKFSISNANIKISGKWKAQKRFLKMSGNFDLSIEGMSISAD
LKLGSNPTSGKPTITASSCSSHINSVHVHISKSKVGWLIQLFHKKIESAL
RNKMNSQVCEKVTNSVSSELQPYFQTLPVMTKIDSVAGINYGLVAPPATT
AETLDVQMKGEFYSENH
CXCL3-BPI:
(SEQ ID NO: 26)
ASVVTELRCQCLQTLQGIHLKNIQSVNVRSPGPHCAQTEVIATLKNGKKA
CLNPASPMVQKIIEKILNKGSTNGGGSGGGSGGGVNPGVVVRISQKGLDY
ASQQGTAALQKELKRIKIPDYSDSFKIKHLGKGHYSFYSMDIREFQLPSS
QISMVPNVGLKFSISNANIKISGKWKAQKRFLKMSGNFDLSIEGMSISAD
LKLGSNPTSGKPTITASSCSSHINSVHVHISKSKVGWLIQLFHKKIESAL
RNKMNSQVCEKVTNSVSSELQPYFQTLPVMTKIDSVAGINYGLVAPPATT
AETLDVQMKGEFYSENH
CXCL5-BPI:
(SEQ ID NO: 27)
LRELRCVCLQTTQGVHPKMISNLQVFAIGPQCSKVEVVASLKNGKEICLD
PEAPFLKKVIQKILDGGNKENGGGSGGGSGGGVNPGVVVRISQKGLDYAS
QQGTAALQKELKRIKIPDYSDSFKIKHLGKGHYSFYSMDIREFQLPSSQI
SMVPNVGLKFSISNANIKISGKWKAQKRFLKMSGNFDLSIEGMSISADLK
LGSNPTSGKPTITASSCSSHINSVHVHISKSKVGWLIQLFHKKIESALRN
KMNSQVCEKVTNSVSSELQPYFQTLPVMTKIDSVAGINYGLVAPPATTAE
TLDVQMKGEFYSENH
CXCL6-BPI:
(SEQ ID NO: 28)
VLTELRCTCLRVTLRVNPKTIGKLQVFPAGPQCSKVEVVASLKNGKQVCL
DPEAPFLKKVIQKILDSGNKKNGGGSGGGSGGGVNPGVVVRISQKGLDYA
SQQGTAALQKELKRIKIPDYSDSFKIKHLGKGHYSFYSMDIREFQLPSSQ
ISMVPNVGLKFSISNANIKISGKWKAQKRFLKMSGNFDLSIEGMSISADL
KLGSNPTSGKPTITASSCSSHINSVHVHISKSKVGWLIQLFHKKIESALR
NKMNSQVCEKVTNSVSSELQPYFQTLPVMTKIDSVAGINYGLVAPPATTA
ETLDVQMKGEFYSENH
CXCL7-BPI:
(SEQ ID NO: 29)
AELRCMCIKTTSGIHPKNIQSLEVIGKGTHCNQVEVIATLKDGRKICLDP
DAPRIKKIVQKKLAGDESADGGGSGGGSGGGVNPGVVVRISQKGLDYASQ
QGTAALQKELKRIKIPDYSDSFKIKHLGKGHYSFYSMDIREFQLPSSQIS
MVPNVGLKFSISNANIKISGKWKAQKRFLKMSGNFDLSIEGMSISADLKL
GSNPTSGKPTITASSCSSHINSVHVHISKSKVGWLIQLFHKKIESALRNK
MNSQVCEKVTNSVSSELQPYFQTLPVMTKIDSVAGINYGLVAPPATTAET
LDVQMKGEFYSENH,
and
CXCL8-BPI:
(SEQ ID NO: 30)
SAKELRCQCIKTYSKPFHPKFIKELRVIESGPHCANTEIIVKLSDGRELC
LDPKENWVQRVVEKFLKRAEGPPSGSGGGSGGGVNPGVVVRISQKGLDYA
SQQGTAALQKELKRIKIPDYSDSFKIKHLGKGHYSFYSMDIREFQLPSSQ
ISMVPNVGLKFSISNANIKISGKWKAQKRFLKMSGNFDLSIEGMSISADL
KLGSNPTSGKPTITASSCSSHINSVHVHISKSKVGWLIQLFHKKIESALR
NKMNSQVCEKVTNSVSSELQPYFQTLPVMTKIDSVAGINYGLVAPPATTA
ETLDVQMKGEFYSENH.

### 2. Efficient Expression Vector of CXCL-BPI Fusion Protein

The designed and optimized coding DNA sequences of CXCL-BPI fusion protein were synthesized by gene synthesis commercial service (Nanjing GenScript Biotech Corporation), and then constructed into the EcoR I/Sal I sites of the pSCm-IL8-BPI eukaryotic expression vector (which was constructed by the inventors and deposited in the China General Microbiological Culture Collection Center on Sep. 15, 2022, with the deposit number of CGMCC NO.: 25726) and were transformed into E. coli JM108, according to conventional technology of molecular cloning. After identification, the CXCL-BPI efficient expression vectors pSCm-CXCL1-BPI, pSCm-CXCL2-BPI, pSCm-CXCL3-BPI, pSCm-CXCL5-BPI, pSCm-CXCL6-BPI, pSCm-CXCL7-BPI and pSCm-CXCL8-BPI (collectively referred to as pSCm-CXCL-BPI) were correctly constructed.

### 3. Stable Transformation, Highly Efficient Expression, Extraction and Preparation of CXCL-BPI Fusion Protein

CHO Grow® CD1 serum-free medium (supplemented with 1×L-alanyl-glutamine solution) (Shanghai BasalMedia Technologies Co., Ltd.) was used to inoculate the suspension-domesticated CHO-K1 cells (ATCC CCL-61) at a density of 3 to 5×10⁵ cells/mL and the cells were cultured for 24 h (37° C., 5% CO₂, 130 rpm). 400 μL of cell suspension (1×10⁷ cells/mL) was collected by centrifugation, mixed with 6 μg of pSCm-CXCL-BPI plasmid (1 μg/μL) well, transferred to 0.8 mL electric shock cup, and electroshocked for twice at 360V and 7 ms (Is interval) by referring to the manual of BTX ECM® 830 electroporation system. The cells were transferred to two 10 cm cell culture dishes (10 mL/dish) and cultured statically for 24 h; then the medium was replaced with a selective medium (CHO Grow® CD1 containing 30 μM MSX), and the cells were inoculated into a 96-well plate at 2×10³ cells/well, and replenished once every 5 to 7 days. After the clones grew to ⅓ of the well area, the efficient expression level of a protein of interest in the supernatant was continuously evaluated (ELISA and SDS-PAGE). The efficiently expressed clones were gradually expanded to a 125 mL shake flask, and the process was screened by protein and cell quality evaluation. Finally, 5 to 10 stable highly expressed clones (expression level at 20 to 60 pcd) were retained for each CXCL-BPI fusion protein.

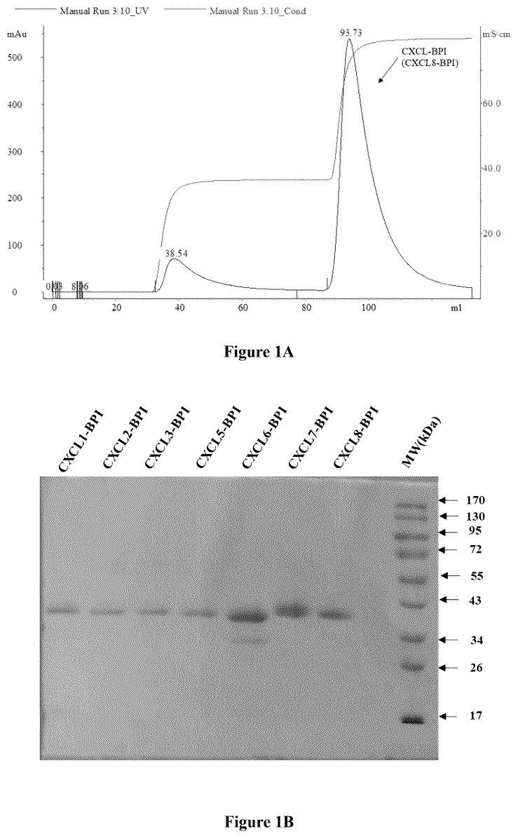

The cells efficiently expressing the CXCL-BPI fusion protein obtained above were inoculated into a cell shake flask (Nalgene™ PETG, 250 mL) at 3×10⁵ cells/mL (the culture medium was CHO Grow® CD1 containing 30 μM MSX), and an appropriate amount of SP Sepharose® Fast Flow was added simultaneously for co-culture (37° C., 5% CO₂, 130 rpm) for 8 to 10 days to capture the protein of interest; SP Sepharose® Fast Flow was collected and loaded into column for purification and preparation by liquid phase chromatography, and subjected to salt concentration gradient elution with 3 mM citrate-13.6 mM phosphate buffer pH6.4 containing 0.10, 0.45 and 1.0 M NaCl, the peak of a typical protein component of interest eluted with 1.0 M NaCl (as shown in FIG. 1A) was collected, replaced with a protein preservation solution (3 mM citrate-13.6 mM phosphate buffer pH6.4 containing 0.5M NaCl), and stored at −30° C. for later use.

The results of SDS-PAGE electrophoresis of the purified protein of interest showed that, as shown in FIG. 1B, each CXCL-BPI fusion protein band was clear (high purity), and at the position consistent with that of the expected molecular weight.

### Example 2: Biological Function of CXCL-BPI Fusion Protein

### 1. Binding to Endotoxin

120 μL of the CXCL-BPI fusion protein of different concentrations (diluted with endotoxin-free PBS, and a blank control was set) and 120 μL of endotoxin (2 EU/mL, diluted with water for endotoxin test) were added to an endotoxin-free glass tube, mixed well by vortexing for 30 s, and placed in a water bath at 37° C. for 1 h; after mixed by vortexing for another 30 s, the mixture was placed in an endotoxin-free 96-well plate at 100 μL/well, and operation was performed according to the instructions of End-point Chromogenic Assay (Xiamen Bioendo Technology Co., Ltd, EC64405).

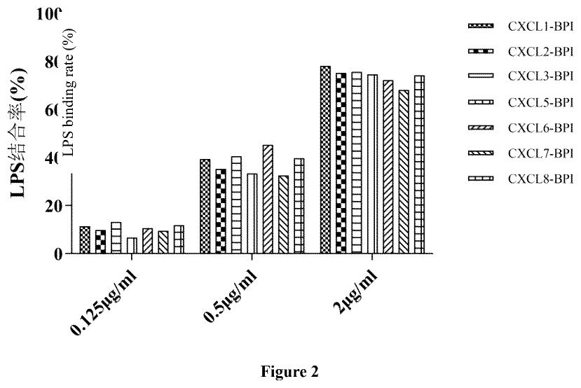

The results showed that, as shown in FIG. 2, the CXCL-BPI fusion protein could neutralize (bind to) LPS in a positively dose-dependent manner.

### 2. Direct Killing of Gram-Negative Bacteria

50 μL of E. coli BL21(DE3)/pBR322 (amp^{R} and tet^{R}) bacterial suspension (1×10⁴ CFU/mL) was mixed with 50 μL of the CXCL-BPI fusion protein of different concentrations (normal saline was used for dilution and as a control), and incubated at 37° C. for 70 min, and 50 μL of each was taken for counting by pouring plate method.

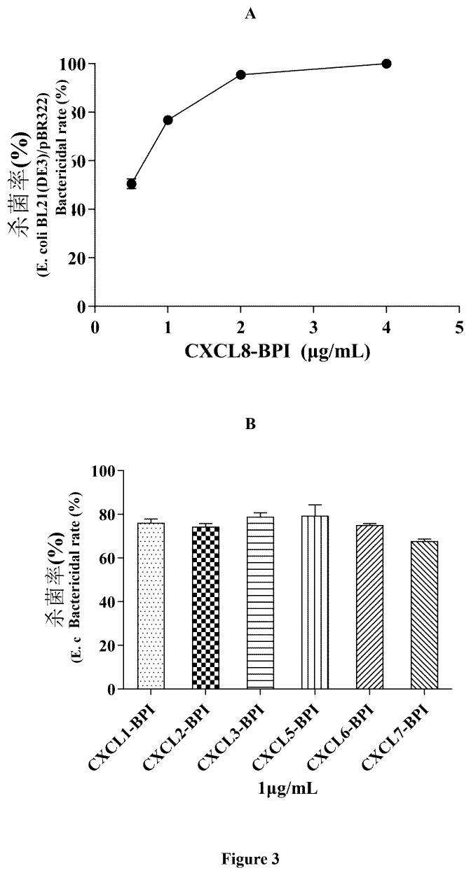

The results showed that, as shown in FIG. 3A, the CXCL8-BPI fusion protein could directly kill Gram-negative bacteria in a positively dose-dependent manner; further, as shown in FIG. 3B, the CXCL1-BPI, CXCL2-BPI, CXCL3-BPI, CXCL5-BPI, CXCL6-BPI and CXCL7-BPI could all directly kill Gram-negative bacteria.

### 3. Inducing Chemotactic Cell Migration

### 3.1 Chemotaxis of Human HL-60 Cells

600 μL/well of the CXCL-BPI fusion protein of different concentrations (diluted with IMDM) was added to the lower chamber of Transwell (Coming, 3422), 1.0×10⁵ cells/100 μL/well of a HL-60 cell suspension (without/with induction differentiation by 1.25% DMSO) was added to the upper chamber thereof, and cultured at 37° C., 8% CO₂ for about 5 hours; the chamber was removed, and the migration of cells was observed under a microscope. Observation method (the same below): 5 areas were randomly selected for each well to take pictures and count (the principle of selecting areas was: upper left, upper right, middle, lower left, and lower right), where the chemotaxis index CI=the number of migrated cells to the sample solution to be tested/the number of migrated cells to the negative control solution.

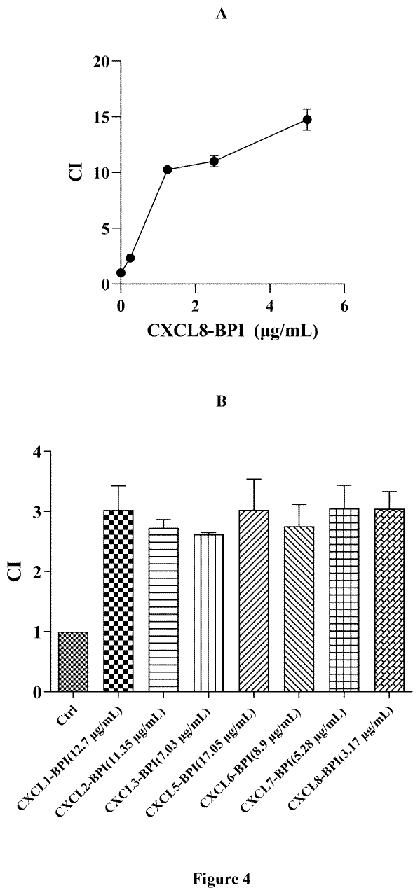

The results showed that, as shown in FIG. 4A, the CXCL8-BPI fusion protein significantly induced chemotaxis of human HL-60 cells (promyelocytes) that had not been differentiated by DMSOs, which was in a positively dose-dependent manner; further, as shown in FIG. 4B, the CXCL-BPI fusion proteins at their respective optimal protein concentrations significantly induced chemotaxis of human HL-60 cells (neutrophil-like cells) that had been differentiated by DMSO.

### 3.2 Chemotaxis of Mouse Bone Marrow Neutrophils

Mice were sacrificed by vertebral dislocation and soaked in 75% ethanol for 5-10 minutes; the tibia and femur were separated, washed in 5 mL PBS, and the muscle tissue was further removed; the ends of the tibia and femur were cut to expose the bone marrow cavity; the bone marrow cavity was rinsed in 8 mL PBS, then fully dispersed, and filtered through a 70 μm nylon mesh; the cells were collected by centrifugation at 3000 rpm for 4 min, resuspended in 3 mL IMDM, and separated by Percoll gradient density centrifugation to prepare neutrophils, and resuspended in 1.3 mL IMDM for later use. Chemotaxis was then carried out by a method same as the experimental method of chemotaxis of HL-60 cells, except that the incubation time at 37° C. and 8% CO₂ was adjusted from 5 h to 2 h.

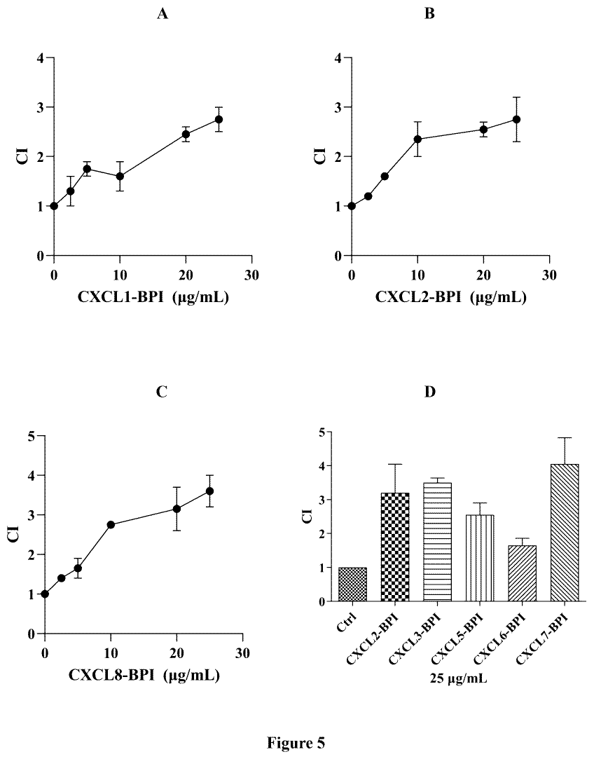

The results showed that, as shown in FIGS. 5A, 5B and 5C, the CXCL1-BPI, CXCL2-BPI and CXCL8-BPI fusion proteins all significantly induced chemotaxis of mouse bone marrow neutrophils in a positively dose-dependent manner; further, as shown in FIG. 5D, the CXCL2-BPI, CXCL3-BPI, CXCL5-BPI, CXCL6-BPI and CXCL7-BPI at a protein concentration of 25 μg/mL all significantly induced chemotaxis of mouse bone marrow neutrophils.

### 3.3 Chemotaxis of Mouse Peritoneal Cells

The mice were sacrificed by vertebral dislocation and soaked in 75% ethanol for 5 minutes; the fur on the abdominal surface was cut to keep the intact peritoneum; 4-5 mL IMDM/mouse was injected into the peritoneal cavity of the mouse and gently massaged for 5 minutes, and the peritoneal fluid was extracted into a 50 mL centrifuge tube (the operation was repeated once); centrifugation was performed at 300 g for 5 minutes, the supernatant was discarded, and the mouse peritoneal cells (containing a large number of phagocytes) were resuspended in 1.3 mL IMDM for later use. Chemotaxis was then carried out by a method same as the experimental method of chemotaxis of HL-60 cells, except that the incubation time at 37° C. and 8% CO₂ was adjusted from 5 hours to 2-2.5 hours.

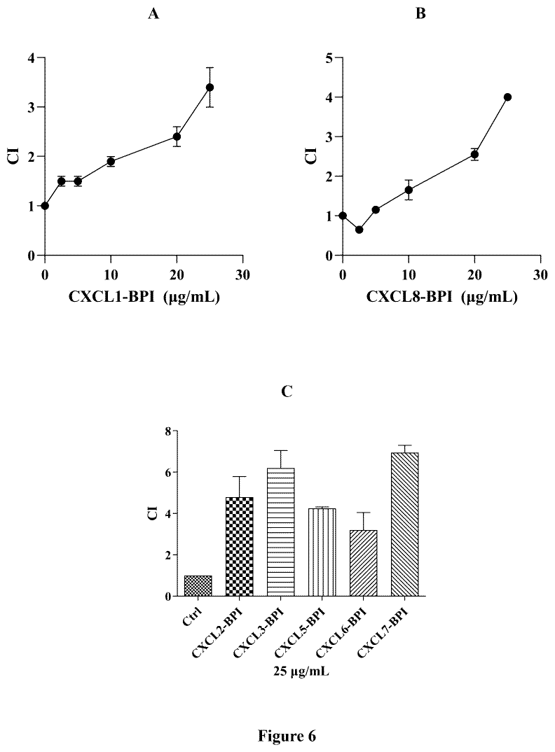

The results showed that, as shown in FIGS. 6A and 6B, both the CXCL1-BPI and CXCL8-BPI fusion proteins significantly induced chemotaxis of mouse peritoneal cells in a positively dose-dependent manner; further, as shown in FIG. 6C, the CXCL2-BPI, CXCL3-BPI, CXCL5-BPI, CXCL6-BPI and CXCL7-BPI, at a protein concentration of 25 μg/mL, all significantly induced chemotaxis of mouse peritoneal cells.

### 4. Promoting Phagocytes to Target, Bind to and Phagocytize Gram-Negative Bacteria

### 4.1 Human HL-60 Cells

HL-60 cells (differentiated into neutrophils by 1.25% DMSO) were stained with DiI (Beyotime, C1036) for 20 min, washed twice with HBSS, resuspended in IMDM to 5×10⁵ cells/mL, added to a 24-well plate at 400 μL/well, then added with 30 μL/well of 1×10⁸ CFU/mL E. coli BL21(DE3)/pET28a-EGFP (kan^{R}) bacterial suspension (the expressed EGFP by IPTG induction was used as a green fluorescent marker; the same below), mixed with the CXCL-BPI fusion protein of different concentrations (a protein diluent was used for dilution and as a control), incubated at 37° C. for 1.5 h, washed twice with HBSS, transferred to a new 24-well plate, and observed under an inverted fluorescence microscope. Observation method (the same below): DiI red fluorescence was used to label cells, EGFP green fluorescence was used to label E. coli BL21(DE3)/pET28a-EGFP, and the two labeled images were superimposed (DiI+EGFP), and the arrows indicated the binding and phagocytosis phenomena (EGFP green fluorescence was observed on the cell membrane and inside the cell).

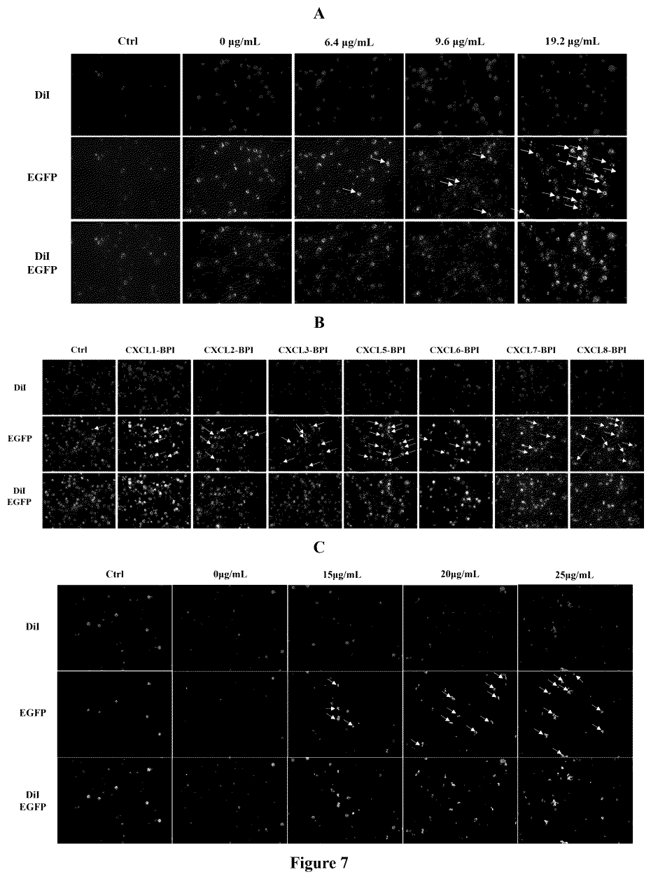

The results showed that, as shown in FIG. 7A, the CXCL8-BPI fusion protein significantly promoted human HL-60 cells (neutrophil-like) to target, bind to and phagocytize Gram-negative bacteria in a positively dose-dependent manner; further, as shown in FIG. 7B, the CXCL-BPI fusion protein significantly promoted human HL-60 cells (neutrophil-like) to target, bind to and phagocytize Gram-negative bacteria at a protein concentration of 20 μg/mL.

### 4.2 Human Peripheral Blood Leukocytes

Human peripheral blood was collected and anticoagulated with 0.4% sodium citrate. Red blood cells were lysed with a red blood cell lysis buffer (Solarbio, R1010). After membrane stained with DiI for 45 minutes, the cells were divided equally according to the experimental group design (about 1×10⁶ cells/group), and collected by centrifugation at 450 g for 5 minutes for later use; 100 μL of E. coli BL21(DE3)/pET28a-EGFP bacterial suspension (washed with PBS and prepared into 2.5×10⁸ CFU/mL bacterial suspension; a negative control was replaced with an equal amount of PBS) was mixed with 100 μL of the preferred CXCL8-BPI fusion protein of different concentrations (both negative and positive controls were replaced by an equal amount of protein diluent) respectively, and incubated at 37° C. for 20 minutes; the cells to be used were respsupended with the suspension of bacteria and protein, added to a 96-well plate, incubated at 37° C. and 200 rpm for 60 minutes, and centrifuged at 450 g for 1 minute, the supernatant was discarded, and the cells were washed once with PBS, fixed with 4% tissue cell fixative (Solarbio, P1110) for 10 minutes, and centrifuged and washed in the same way, and the cells were resuspended in an appropriate amount of an anti-fluorescence quencher, spotted, sealed, and observed under a fluorescence microscope.

The results showed that, as shown in FIG. 7C, the preferred CXCL8-BPI fusion protein significantly promoted human peripheral blood phagocytes (mainly neutrophils, followed by monocytes) to target, bind to and phagocytize Gram-negative bacteria in a positively dose-dependent manner.

### 4.3 Mouse Peripheral Blood Leukocytes

Blood was collected from the mouse mandible, anticoagulated with 0.4% sodium citrate, and lysed with an erythrocyte lysis buffer; and the experiment was conducted with 20 μg/mL CXCL-BPI fusion protein by referring to the experimental method for human HL-60 cells in 4.1.

The results showed that, as shown in FIG. 7D, the CXCL-BPI fusion protein significantly promoted the mouse peripheral blood phagocytes (mainly neutrophils, followed by monocytes) to target, bind to and phagocytize Gram-negative bacteria.

### 4.4 Mouse Peritoneal Phagocytes

Mouse peritoneal cells were prepared according to the experimental method in 3.3, resuspended in an appropriate amount of DMEM-H, inoculated in a 24-well plate at 100 μL/well, and cultured at 37° C. and 8% CO₂ to allow the cells to adhere to the wall; after the cells were stained with DiI, the experiment was conducted with different concentrations of the preferred CXCL8-BPI fusion protein by referring to the experimental method for human peripheral blood leukocytes in 4.2, and the difference was that the peritoneal phagocytes were in an adherent state, and no centrifugation was required during the operation.

The results showed that, as shown in FIG. 7E, the preferred CXCL8-BPI fusion protein significantly promoted mouse peritoneal phagocytes (including macrophages and neutrophils) to target, bind to and phagocytize Gram-negative bacteria in a positively dose-dependent manner.

### Example 3: Bactericidal Effect of CXCL-BPI Fusion Protein in Peripheral Blood and Peritoneal Phagocytes

In view of the strong rejection of human peripheral blood (healthy volunteers) to E. coli BL21 (DE3) (e.g., serotype-specific humoral immunity and phagocytic clearance), Acinetobacter baumannii was selected for bactericidal assay in human peripheral blood in this example, while Acinetobacter baumannii and E. coli BL21 (DE3) could be selected for bactericidal assays in mouse peripheral blood and mouse peritoneal phagocytes.

### 1. Bactericidal Assay in Human/Mouse Peripheral Blood

100 μL of a bacterial suspension of Acinetobacter baumannii (ATCC BAA-1605, Multidrug-resistant) (2×10⁴ CFU/mL in normal saline), 100 μL of the CXCL-BPI fusion protein of different concentrations (diluted in normal saline), 180 μL of normal saline and 20 μL of human/mouse peripheral blood (anticoagulated with 0.4% sodium citrate) were mixed well. In the experiment, a heat-treated human/mouse peripheral blood (heat treatment in a water bath at 56° C. for 30 min to inhibit or destroy relevant biological activities of phagocytes and complements etc.) was set as a control group, and incubated at 37° C. for 1 h. 50 μL of each sample was taken for counting by pouring plate method.

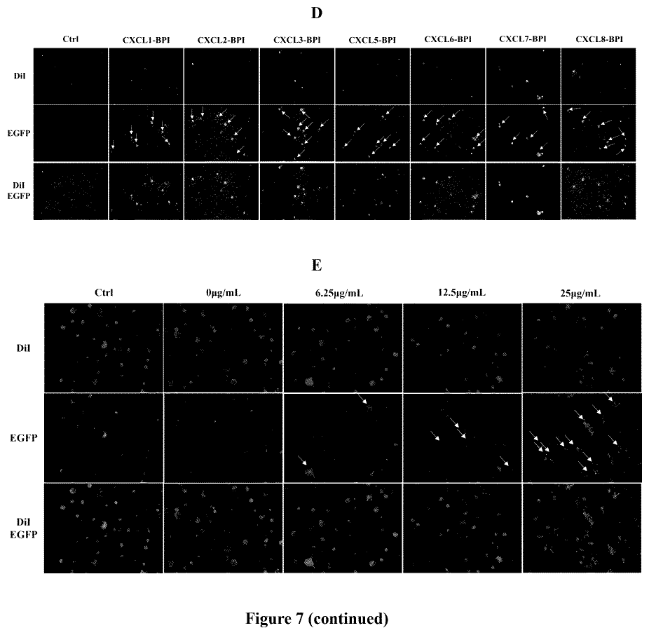

The results showed that, as shown in FIG. 8A/8C, within a relatively low concentration range, the preferred CXCL8-BPI fusion protein had a significantly higher bactericidal effect in human/mouse peripheral blood as compared to the heat-treated human/mouse peripheral blood control group, and the difference was negatively correlated with the concentration (i.e., the lower the concentration, the more significant the difference), suggesting that the fusion protein promoted the peripheral blood phagocytes to target, bind to and phagocytize Gram-negative bacteria; further, as shown in FIG. 8B/8D, the CXCL1-BPI, CXCL2-BPI, CXCL3-BPI, CXCL5-BPI, CXCL6-BPI and CXCL7-BPI had significantly higher bactericidal effects in human/mouse peripheral blood as compared to the heat-treated human/mouse peripheral blood control group at their respective optimal protein concentrations.

### 2. Bactericidal Assay in Mouse Peripheral Blood

By referring to the experimental method in the item 1 above, 100 μL of a bacterial suspension of E. coli BL21(DE3)/pBR322 (1×10⁴ CFU/mL), 100 μL of the CXCL8-BPI fusion protein of different concentrations (a protein diluent was used for dilution and as a control), and 40 ul of mouse peripheral blood (0.4% sodium citrate for anticoagulation) were mixed well, and incubated at 37° C. for 1.5 h; and 100 μL of each sample was taken for counting by pouring plate method.

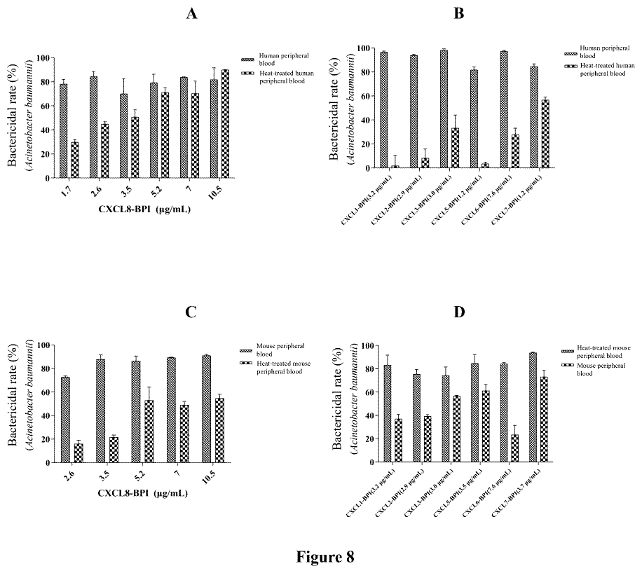

The results showed that, as shown in FIG. 9A, the preferred CXCL8-BPI fusion protein had a significant bactericidal effect in mouse peripheral blood in a positively dose-dependent manner; further, as shown in FIG. 9B, the CXCL1-BPI, CXCL2-BPI, CXCL3-BPI, CXCL5-BPI, CXCL6-BPI and CXCL7-BPI fusion proteins all had a significant bactericidal effect in mouse peripheral blood.

### 3. Bactericidal Assay in Mouse Peritoneal Phagocytes

Mouse peritoneal cells were prepared according to the experimental method in Section 3.3 of Example 2, resuspended in an appropriate amount of IMDM, spread into a 96-well plate at 100 μL/well, and cultured in a 37° C., 8% CO₂ incubator for about 4 hours until the cells adhered to the wall and the confluence was about 80%; IMDM (cell-free group) was set as a control in the experiment; washing was performed once with 200 μL/well of saline; 1×10⁴ CFU/mL of the bacterial suspension of E. coli BL21(DE3)/pBR322 was taken and mixed with equal volumes of the preferred CXCL8-BPI fusion protein at different concentrations (normal saline was used for dilution and as a control), respectively, incubated at 37° C. for 10 min, and added to the above-mentioned wells of cell at 100 μL/well, and then the 96-well plate was incubated at 37° C. for 60 min; and 50 μL of each sample was taken for counting by pouring plate method. In addition, an experiment same as that above was carried out, and CHO-DG44 cells (non-phagocytic cell group) were set as a control.

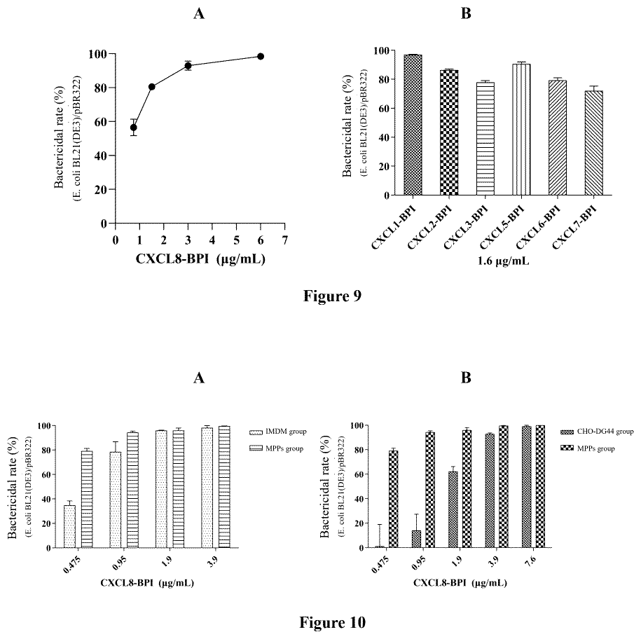

The results showed that, as shown in FIGS. 10A and 10B, within a relatively low concentration range, the preferred CXCL8-BPI fusion protein had a significantly higher bactericidal effect in mouse peritoneal phagocytes (MPPs) as compared to the cell-free control group and the CHO-DG44 cell control group, and the difference was negatively correlated with the concentration (i.e., the lower the concentration, the more significant the difference), indicating that the fusion protein promoted peritoneal phagocytes to target, bind to and phagocytize Gram-negative bacteria.

### Example 4: Protective Effect of CXCL-BPI Fusion Protein on Mice Infected with Gram-Negative Bacteria

1. Mouse Model Infected with Gram-Negative Bacteria

Mouse infection model (dose): E. coli BL21(DE3)/pBR322 was diluted with PBS into bacterial suspensions of different concentrations, and the bacterial suspensions (0.25 mL/mouse) were intraperitoneally injected into 6-8 week-old mice randomly divided into groups (5 mice per group). At 3, 6, 9, 12 and 24 hours, one mouse was taken from each group for blood sampling by retro-orbital bleeding, the blood sample was stood for 40 minutes and centrifuged at 1000 rpm for 10 minutes, and serum was diluted 10 times with saline. 50 μL of each sample was taken for counting by pouring plate method (repeated with 2 dishes, the same below). The dynamic changes of bacterial count in the serum under different intraperitoneal injection bacterial amounts were statistically observed, and at the same time, the dynamic changes of coat color, activity, diarrhea and other states were observed and recorded. The amount of injected bacteria at which the mice showed a serum bacterial load suitable for observation and obvious infection symptoms was determined as a dose in subsequent in vivo assay.

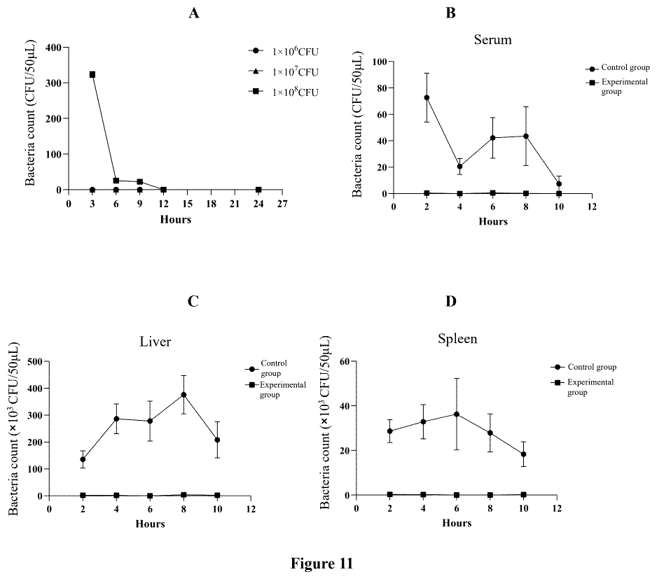

The results showed that, as shown in FIG. 11A, 1×10⁸ CFU/mouse was a suitable dose for mouse intra-abdominal infection model in vivo assay.

2. Protective Effect of CXCL-BPI Fusion Protein on Mice Infected with Gram-Negative Bacteria

The CXCL8-BPI fusion protein was preferred for this assay. Randomly grouped 6-8-week-old mice (BALB/c) were intraperitoneally injected with 1×10⁸ CFU/0.25 mL/mouse of a bacterial suspension of E. coli BL21(DE3)/pBR322 for infection and challenge. 10 minutes later, the preferred CXCL8-BPI fusion protein (0.3 mg/0.25 mL/mouse, in the control group, it was replaced with the corresponding buffer) was intraperitoneally injected into the mice. At 2, 4, 6, 8 and 10 hours, 6 to 7 mice were taken from each group, respectively, and treated as follows: 1) blood was collected from the eyeball, stood for 40 minutes, and centrifuged at 1000 rpm for 10 minutes, and serum was diluted 10 times with normal saline to prepare serum samples. 50 μL of each sample was taken for counting by pouring plate method; 2) organs (liver and spleen) were separated, rinsed with an appropriate amount of sterile saline, ground, resuspended in 3 mL sterile saline, filtered through a 70 μm mesh to prepare homogenate specimens, and 50 μL of each sample was taken for counting by pouring method. The dynamic changes of bacterial counts in the serum and organs of mice in each group were statistically observed, and the dynamic changes of their activity, fur color, diarrhea and other states were observed and recorded.

The results showed that, as shown in FIGS. 11B, C, D and Table 3, the preferred CXCL8-BPI fusion protein had a significant protective effect on infected mice in vivo. The bacterial counts in the serum and organs (liver and spleen) of mice in the test groups were significantly lower than those in the control group. At the same time, the state of the mice in the test groups (significantly better, substantially no obvious diarrhea) was significantly better than that in the control group (activity was severely reduced, fur was frizzy and most of them were accompanied by diarrhea at 4 to 8 hours, and most of these symptoms were relieved after 10 hours).

TABLE 3
Protective effect of CXCL8-BPI on mice infected with E. coli BL21(DE3)/pBR322

| | | | | Experimental group | | |
|---|---|---|---|---|---|---|
| | | Control group | | (0.3 mg/animal) | | |
| Sample | Sampling | Number | Bacterial count | Number | Bacterial count | |
| type | time (h) | of mice | (CFU/50 μL) | of mice | (CFU/50 μL) | t test |
| Serum | 2 | 6 | 72.7 ± 18.5 | 7 | 0.4 ± 0.2 | * |
| | 4 | 6 | 20.5 ± 6.1 | 7 | 0.0 ± 0.0 | P = 0.0288 |
| | 6 | 6 | 42.2 ± 15.4 | 6 | 0.5 ± 0.2 | |
| | 8 | 6 | 43.5 ± 22.3 | 6 | 0.2 ± 0.2 | |
| | 10 | 6 | 7.3 ± 6.0 | 6 | 0.0 ± 0.0 | |
| Liver | 2 | 6 | (135.7 ± 32.3) × 10³ | 7 | (2.4 ± 1.4) × 10³ | *** |
| | 4 | 6 | (286.3 ± 55.6) × 10³ | 7 | (2.0 ± 0.9) × 10³ | P = 0.0032 |
| | 6 | 6 | (278.0 ± 74.1) × 10³ | 6 | (0.3 ± 0.1) × 10³ | |
| | 8 | 6 | (376.0 ± 71.1) × 10³ | 6 | (4.1 ± 3.6) × 10³ | |
| | 10 | 6 | (208.0 ± 67.1) × 10³ | 6 | (2.4 ± 1.0) × 10³ | |
| Spleen | 2 | 6 | (28.7 ± 5.1) × 10³ | 7 | (0.3 ± 0.1) × 10³ | *** |
| | 4 | 6 | (32.9 ± 7.7) × 10³ | 7 | (0.3 ± 0.1) × 10³ | P = 0.0007 |
| | 6 | 6 | (36.3 ± 16.1) × 10³ | 6 | (0.1 ± 0.0) × 10³ | |
| | 8 | 6 | (27.9 ± 8.5) × 10³ | 6 | (0.1 ± 0.0) × 10³ | |
| | 10 | 6 | (18.3 ± 5.6) × 10³ | 6 | (0.2 ± 0.1) × 10³ | |

1. A CXCL-BPI fusion protein, comprising a human ELR+CXC chemokine and a bioactive fragment of N-terminal domain of human BPI.

2. The CXCL-BPI fusion protein according to claim 1, wherein the human ELR+CXC chemokine is selected from the group consisting of human CXCL1, human CXCL2, human CXCL3, human CXCL5, human CXCL6, human CXCL7 and human CXCL8, and optionally, the human CXCL8, human CXCL1, human CXCL2, human CXCL3, human CXCL5, human CXCL6 or human CXCL7 comprises the sequence as set forth in SEQ ID NO: 1, SEQ ID NO: 2, SEQ ID NO: 3, SEQ ID NO: 4, SEQ ID NO: 5, SEQ ID NO: 6 or SEQ ID NO: 7, respectively.

3. The CXCL-BPI fusion protein according to claim 1, wherein the bioactive fragment of N-terminal domain of human BPI is selected from the group consisting of human BPI₁₋₂₃₃, BPI₁₋₁₉₉, and BPI₁₋₁₉₃, and optionally, the human BPI₁₋₂₃₃ comprises the sequence as set forth in SEQ ID NO: 10.

4. The CXCL-BPI fusion protein according to claim 1, wherein the human ELR+CXC chemokine is used as the N-terminal domain of the fusion protein, and the bioactive fragment of N-terminal domain of human BPI is used as the C-terminal domain of the fusion protein, and the two are optionally connected via a linker, and further optionally, the linker is selected from GPPSGSGGGSGGG (SEQ ID NO: 8) and GGGSGGGSGGG (SEQ ID NO: 9).

5. A nucleic acid, encoding the CXCL-BPI fusion protein according to claim 1.

6. The nucleic acid according claim 5, which comprises, from 5′ end to 3′ end, a 5′ adapter sequence, a signal peptide coding sequence, a human ELR+CXC chemokine coding sequence, a linker coding sequence, a coding sequence of bioactive fragment of N-terminal domain of human BPI and a 3′ adapter sequence in sequence, optionally, wherein the human ELR+CXC chemokine coding sequence comprises the sequence as set forth in SEQ ID NO: 13, SEQ ID NO: 14, SEQ ID NO: 15, SEQ ID NO: 16, SEQ ID NO: 17, SEQ ID NO: 18 or SEQ ID NO: 19, respectively, and the coding sequence of bioactive fragment of N-terminal domain of human BPI comprises the sequence as set forth in SEQ ID NO: 22.

7. (canceled)

8. An expression vector wherein the expression vector comprises a nucleic acid encoding the CXCL-BPI fusion protein according to claim 1.

9. The expression vector according to claim 8, which is selected from efficient expression vectors pSCm-CXCL1-BPI, pSCm-CXCL2-BPI, pSCm-CXCL3-BPI, pSCm-CXCL5-BPI, pSCm-CXCL6-BPI, pSCm-CXCL7-BPI and pSCm-CXCL8-BPI.

10. A pharmaceutical composition, comprising the CXCL-BPI fusion protein according to claim 1 and a pharmaceutically acceptable diluent, adjuvant, or carrier.

11. A host cell, comprising an expression vector, wherein the expression vector is capable of performing stable transfection or transformation with a nucleic acid encoding the CXCL-BPI fusion protein according to claim 1.

12. A method for preparing a CXCL-BPI fusion protein, comprising culturing the host cell according to claim 11 under conditions suitable for the expression of the CXCL-BPI fusion protein, harvesting the expressed CXCL-BPI fusion protein, and optionally further purifying the expressed CXCL-BPI fusion protein.

13. A method for treating Gram-negative bacterial infection, comprising administering a therapeutically effective amount of the pharmaceutical composition according to claim 10 to a subject suffering from a Gram-negative bacterial infection.

14. (canceled)

15. The CXCL-BPI fusion protein according to claim 1, wherein the human ELR+CXC chemokine is selected from the group consisting of human CXCL1, human CXCL2, human CXCL3, human CXCL5, human CXCL6, human CXCL7 and human CXCL8, and optionally, the human CXCL8, human CXCL1, human CXCL2, human CXCL3, human CXCL5, human CXCL6 or human CXCL7 comprises the sequence as set forth in SEQ ID NO: 1, SEQ ID NO: 2, SEQ ID NO: 3, SEQ ID NO: 4, SEQ ID NO: 5, SEQ ID NO: 6 or SEQ ID NO: 7, respectively; wherein the bioactive fragment of N-terminal domain of human BPI is selected from the group consisting of human BPI₁₋₂₃₃, human BPI₁₋₉₉ and human BPI₁₋₁₉₃, and optionally, the human BPI₁₋₂₃₃ comprises the sequence as set forth in SEQ ID NO: 10; and wherein the human ELR+CXC chemokine is used as the N-terminal domain of the fusion protein, and the bioactive fragment of N-terminal domain of human BPI is used as the C-terminal domain of the fusion protein, and the two are optionally connected via a linker, and further optionally, the linker is selected from GPPSGSGGGSGGG (SEQ ID NO: 8) and GGGSGGGSGGG (SEQ ID NO: 9).


SEQUENCE LISTING (30 sequences)

SEQ ID NO: 1 - protein, 70 aa, synthetic construct
SAKELRCQCIKTYSKPFHPKFIKELRVIESGPHCANTEIIVKLSDGRELCLDPKENWVQRVVEKFLKRAE

SEQ ID NO: 2 - protein, 73 aa, synthetic construct
ASVATELRCQCLQTLQGIHPKNIQSVNVKSPGPHCAQTEVIATLKNGRKACLNPASPIVKKIIEKMLNSDKSN

SEQ ID NO: 3 - protein, 73 aa, synthetic construct
APLATELRCQCLQTLQGIHLKNIQSVKVKSPGPHCAQTEVIATLKNGQKACLNPASPMVKKIIEKMLKNGKSN

SEQ ID NO: 4 - protein, 73 aa, synthetic construct
ASVVTELRCQCLQTLQGIHLKNIQSVNVRSPGPHCAQTEVIATLKNGKKACLNPASPMVQKIIEKILNKGSTN

SEQ ID NO: 5 - protein, 71 aa, synthetic construct
LRELRCVCLQTTQGVHPKMISNLQVFAIGPQCSKVEVVASLKNGKEICLDPEAPFLKKVIQKILDGGNKEN

SEQ ID NO: 6 - protein, 72 aa, synthetic construct
VLTELRCTCLRVTLRVNPKTIGKLQVFPAGPQCSKVEVVASLKNGKQVCLDPEAPFLKKVIQKILDSGNKKN

SEQ ID NO: 7 - protein, 70 aa, synthetic construct
AELRCMCIKTTSGIHPKNIQSLEVIGKGTHCNQVEVIATLKDGRKICLDPDAPRIKKIVQKKLAGDESAD

SEQ ID NO: 8 - protein, 13 aa, synthetic construct
GPPSGSGGGSGGG

SEQ ID NO: 9 - protein, 11 aa, synthetic construct
GGGSGGGSGGG

SEQ ID NO: 10 - protein, 233 aa, synthetic construct
VNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 11 - DNA, 12 bp, synthetic construct
  source 1..12: mol_type = other DNA
gaattcgccacc

SEQ ID NO: 12 - DNA, 57 bp, synthetic construct
  source 1..57: mol_type = other DNA
atgggatggagttgcatcatcctgtttctggtggctaccgcaacaggcgtgcattct

SEQ ID NO: 13 - DNA, 210 bp, synthetic construct
  source 1..210: mol_type = other DNA
tctgccaaggaactgcggtgtcagtgcatcaagacctacagcaagcctttccaccccaagttcatcaaagaactgagagtgatcgagtccggccctcactgtgccaacaccgagatcatcgtgaagctgtccgacggccgcgagctgtgcctggaccctaaggaaaattgggtgcagagagtcgtggaaaagtttctgaagagagccgag

SEQ ID NO: 14 - DNA, 219 bp, synthetic construct
  source 1..219: mol_type = other DNA
gcttctgtcgccaccgagctgcggtgccagtgcctgcagaccctgcagggcatccaccccaagaacatccagtccgtgaacgtgaaatctcctggccctcactgcgcccagaccgaagtgatcgccacactgaagaacggccggaaggcctgtctgaaccctgcctctccaatcgtgaagaagatcatcgagaagatgctgaactccgacaagtccaac

SEQ ID NO: 15 - DNA, 219 bp, synthetic construct
  source 1..219: mol_type = other DNA
gctcctctggctaccgagctgcggtgccagtgcctgcagaccctgcagggcatccacctgaagaacatccagtccgtgaaagtgaagtctccaggccctcactgcgcccagaccgaagtgatcgccacactgaagaacggacagaaggcctgtctgaaccccgcctcccctatggtcaagaagatcatcgagaagatgctgaaaaacggcaagtccaac

SEQ ID NO: 16 - DNA, 219 bp, synthetic construct
  source 1..219: mol_type = other DNA
gcttctgtcgtgaccgagctgagatgccagtgcctgcagacactgcagggcatccacctgaaaaacatccagtccgtgaacgtgcggtctccaggacctcactgcgcccagaccgaagtgatcgccaccctgaagaacggcaagaaggcctgtctgaatcctgctagccctatggtgcagaagatcatcgagaagatcctgaacaagggctccaccaac

SEQ ID NO: 17 - DNA, 213 bp, synthetic construct
  source 1..213: mol_type = other DNA
ctgagagagctgcggtgcgtgtgtctgcagaccacacagggcgtgcaccctaagatgatctccaacctgcaggtgttcgccatcggccctcagtgctccaaggtggaagtggtggcctccctgaagaacggcaaggagatctgcctggaccctgaggcccctttcctgaagaaagtgatccagaagatcctggacggcggcaacaaggaaaat

SEQ ID NO: 18 - DNA, 216 bp, synthetic construct
  source 1..216: mol_type = other DNA
gtgctgaccgagctgagatgcacctgtctgagagtgaccctgcgggtgaaccccaagaccatcggcaagctgcaggtgttccctgctggccctcagtgctccaaggtggaagtggtggcctctctgaagaacggcaaacaggtgtgcctggaccctgaggctcctttcctgaagaaagtgatccagaagatcctggactccggcaacaagaagaat

SEQ ID NO: 19 - DNA, 210 bp, synthetic construct
  source 1..210: mol_type = other DNA
gccgagctgagatgcatgtgcatcaagaccacctctggcatccatcccaagaacatccagtccctggaagtcatcggcaagggaacccactgcaaccaggtggaagtgatcgccacactgaaggacggcagaaagatctgcctggaccctgacgctcctcggatcaagaaaatcgtgcagaagaagctggctggcgacgagtccgctgat

SEQ ID NO: 20 - DNA, 39 bp, synthetic construct
  source 1..39: mol_type = other DNA
ggacctccaagtggcagtggtggcggatccggtggcggt

SEQ ID NO: 21 - DNA, 33 bp, synthetic construct
  source 1..33: mol_type = other DNA
ggtggaggcagtggtggcggatccggtggcggt

SEQ ID NO: 22 - DNA, 699 bp, synthetic construct
  source 1..699: mol_type = other DNA
gtgaaccccggcgtggtggtgcggatctcccagaaaggcctggattacgccagccagcagggaacagctgctctgcagaaagaactgaaaagaatcaagatcccagactacagtgatagcttcaagatcaagcacctgggaaagggccactactctttctactctatggacatcagagagtttcagctgccctcctctcagatctctatggtgcctaacgtgggcctgaagttcagcatctccaacgccaatatcaagatttctggcaagtggaaggctcagaagcggttcctgaagatgtctggcaacttcgacctgtccatcgaaggcatgtccatcagcgccgatctgaagctgggctctaatcctacctccggcaagcccacaatcaccgcctctagctgttcctctcacatcaacagcgtgcacgtgcacatctctaagtccaaagtgggctggctgatccagctgttccacaagaagatcgagtctgctctgcggaacaagatgaactctcaggtgtgcgagaaggtgaccaactccgtgtccagcgaactccagccttatttccagaccctgcccgtgatgaccaagatcgactccgtggctggcatcaactacggcctggtggccccacctgctactaccgccgagacactggacgtgcagatgaagggcgagttctactccgagaaccac

SEQ ID NO: 23 - skipped

SEQ ID NO: 24 - protein, 317 aa, synthetic construct
ASVATELRCQCLQTLQGIHPKNIQSVNVKSPGPHCAQTEVIATLKNGRKACLNPASPIVKKIIEKMLNSDKSNGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 25 - protein, 317 aa, synthetic construct
APLATELRCQCLQTLQGIHLKNIQSVKVKSPGPHCAQTEVIATLKNGQKACLNPASPMVKKIIEKMLKNGKSNGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 26 - protein, 317 aa, synthetic construct
ASVVTELRCQCLQTLQGIHLKNIQSVNVRSPGPHCAQTEVIATLKNGKKACLNPASPMVQKIIEKILNKGSTNGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 27 - protein, 315 aa, synthetic construct
LRELRCVCLQTTQGVHPKMISNLQVFAIGPQCSKVEVVASLKNGKEICLDPEAPFLKKVIQKILDGGNKENGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 28 - protein, 316 aa, synthetic construct
VLTELRCTCLRVTLRVNPKTIGKLQVFPAGPQCSKVEVVASLKNGKQVCLDPEAPFLKKVIQKILDSGNKKNGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 29 - protein, 314 aa, synthetic construct
AELRCMCIKTTSGIHPKNIQSLEVIGKGTHCNQVEVIATLKDGRKICLDPDAPRIKKIVQKKLAGDESADGGGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH

SEQ ID NO: 30 - protein, 316 aa, synthetic construct
SAKELRCQCIKTYSKPFHPKFIKELRVIESGPHCANTEIIVKLSDGRELCLDPKENWVQRVVEKFLKRAEGPPSGSGGGSGGGVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITASSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENH